(19) United States
(12) Patent Application Publication
(10) Pub. No.: US 2023/0348533 A1
(43) Pub. Date: Nov. 2, 2023

(54) BIOANALYSIS OF THERAPEUTIC ANTIBODIES AND RELATED PRODUCTS USING IMMUNOPRECIPITATION AND NATIVE SEC-PCD-MS DETECTION

(71) Applicant: Regeneron Pharmaceuticals, Inc., Tarrytown, NY (US)

(72) Inventors: Yuetian Yan, Chappaqua, NY (US); Shunhai Wang, Scarsdale, NY (US)

(21) Appl. No.: 18/197,579

(22) Filed: May 15, 2023

Related U.S. Application Data
(63) Continuation-in-part of application No. 17/863,332, filed on Jul. 12, 2022.
(60) Provisional application No. 63/221,439, filed on Jul. 13, 2021.

(51) Int. Cl. C07K 1/36

(52) U.S. Cl. CPC C07K 1/36

(57) ABSTRACT

The present invention generally pertains to methods of characterizing antibodies and related products. In particular, the present invention pertains to the use of immunoprecipitation and postcolumn denaturation-assisted native size exclusion chromatography-mass spectrometry to specifically and sensitively detected and quantitate antibodies and related products in a sample.

### CROSS-REFERENCE TO RELATED APPLICATIONS

This application is a continuation-in-part of U.S. patent application Ser. No. 17/863,332, filed on Jul. 12, 2022, which claims priority to and the benefit of U.S. Provisional Patent Application No. 63/221,439, filed on Jul. 13, 2021, which is herein incorporated by reference.

### FIELD

The invention generally relates to methods for characterizing antibodies and related products.

### BACKGROUND

Product quality attributes of therapeutic peptides or proteins are extensively characterized to ensure preservation of their associated safety, efficacy, and shelf life profiles relevant to pharmacokinetics. Alterations of therapeutic peptides or proteins may occur at any point during and after the production and/or purification process. The therapeutic peptides or proteins can become heterogeneous due to various post-translational modifications, protein degradation, enzymatic modifications, and chemical modifications.

Other key features of a therapeutic peptide or protein include properties such as pharmacokinetics and pharmacodynamics that determine the abundance and timing of the therapy in vivo. Understanding the processing of a therapeutic in vivo can be essential to determining how that therapeutic is best produced and delivered, for example determining routes of administration, dosing, and therapeutic and adverse effects.

Accurately and efficiently assessing these features of a therapeutic peptide or protein, often in the context of a complex matrix such as serum that complicates detection, requires high-throughput, high-sensitivity and high-specificity techniques. It will be appreciated that a need exists for methods and systems to achieve accurate characterization and quantitation of therapeutic peptides and proteins and their key features.

### SUMMARY

A native strong cation exchange-mass spectrometry (SCX-MS) method has been developed for the detection and quantitation of antibodies and related products. Immunoprecipitation with agarose beads coated in anti-human Fc antibody may be used to pull down a human antibody in a sample. The digestive enzyme IdeS or a variant thereof may be used to cleave the immobilized antibody, producing a Fab₂ fragment that may be eluted and collected. This fragment may then be subjected to native SCX-MS analysis for sensitive and robust quantitation. The method of the present invention was shown to efficiently and accurately quantitate antibodies even at low concentrations, in neat solution or in serum, as demonstrated in the Examples.

Additionally, a postcolumn denaturation-assisted native size exclusion chromatography-mass spectrometry (nSEC-PCD-MS) method has been developed for identifying, quantifying, and characterizing therapeutic proteins, biotransformation products, and interacting proteins. Proteins may be enriched from a sample using immunoprecipitation and then subjected to SEC separation. Denaturing solution may be introduced into the SEC eluate flow to separate non-covalently bound complexes. The denatured eluate can then be subjected to MS analysis to characterize the protein, variants thereof, and any interacting proteins, including over periods of time after administration to a subject for pharmacokinetic (PK) analysis.

This disclosure provides a method for characterization of an antibody. In some exemplary embodiments, the method comprises: (a) immobilizing said antibody on a solid-phase substrate; (b) contacting said immobilized antibody to a digestive enzyme to produce an unbound fragment of said antibody; (c) eluting said antibody fragment; and (d) subjecting said eluate to native SCX-MS analysis to characterize said antibody.

In one aspect, said antibody is a monoclonal antibody or a bispecific antibody.

In one aspect, said immobilizing step comprises contacting a sample including said antibody to a solid-phase substrate capable of binding to said antibody. In a specific aspect, said sample is a serum sample.

In one aspect, said solid-phase substrate comprises beads. In a specific aspect, said beads are agarose beads or magnetic beads.

In a specific aspect, said binding of said solid-phase substrate is performed by an antibody adhered to said solid-phase substrate. In a further specific aspect, said antibody is an anti-Fc antibody.

In one aspect, the method further comprises a step of washing said solid-phase substrate after immobilizing said antibody.

In one aspect, said digestive enzyme is IdeS or a variant thereof. In another aspect, said antibody fragment is a Fab₂ fragment.

In one aspect, said eluting comprises a step of centrifuging said solid-phase substrate and antibody fragment.

In one aspect, said SCX system is coupled to said mass spectrometer. In another aspect, said mass spectrometer is an electrospray ionization mass spectrometer, nano-electrospray ionization mass spectrometer, or a triple quadrupole mass spectrometer.

In one aspect, said characterization of an antibody comprises quantitation of an antibody, optionally wherein said quantitation is normalized to an internal standard.

This disclosure also provides methods for identifying, quantifying, and/or characterizing a protein of interest. In some exemplary embodiments, the methods can comprise (a) contacting a sample including a protein of interest to a solid surface to form an immobilized protein of interest; (b) eluting said immobilized protein of interest or a fragment thereof to form an enriched protein of interest or fragment thereof; (c) subjecting said enriched protein of interest or fragment thereof to size exclusion chromatography under native conditions to form a size exclusion chromatography eluate; (d) contacting said size exclusion chromatography eluate to a denaturing solution to form a denatured eluate; and (e) subjecting said denatured eluate to mass spectrometry analysis to identify, quantify, and/or characterize said protein of interest.

In one aspect, said sample is selected from a group consisting of cell culture fluid, harvested cell culture fluid, drug substance, drug product, a tissue sample, blood, serum, saliva, or urine. In a specific aspect, said sample is human serum or mouse serum.

In one aspect, said protein of interest is selected from a group consisting of a recombinant protein, a therapeutic protein, an antibody, a bispecific antibody, a trispecific antibody, a multispecific antibody, an antibody fragment, a fusion protein, a trap protein, a single-chain variable fragment, and combinations thereof.

In one aspect, said protein of interest is a therapeutic antibody.

In one aspect, said solid surface is selected from a group consisting of a microplate, resin, agarose beads, or magnetic beads.

In one aspect, said solid surface is adhered to an antibody that can specifically bind to said protein of interest. In a specific aspect, said adhering is mediated by biotin and avidin or streptavidin. In another specific aspect, said antibody is an anti-Fc antibody.

In one aspect, the methods can further comprise subjecting said immobilized protein of interest to at least one washing step to remove non-specifically bound components.

In one aspect, the methods can further comprise contacting said immobilized protein of interest to at least one digestive enzyme. In a specific aspect, said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease, LysC endoproteinase, endoproteinase AspN, endoproteinase GluC, outer membrane protein T, immunoglobulin-degrading enzyme of Streptococcus pyogenes (IdeS), thermolysin, papain, pronase, V8 protease, active fragments thereof, homologs thereof, variants thereof, and combinations thereof. In a more specific aspect, said at least one digestive enzyme comprises IdeS or a variant thereof.

In one aspect, eluting said immobilized protein of interest or fragment thereof comprises subjecting said solid surface to centrifugation.

In one aspect, said fragment is selected from the group consisting of a Fab fragment, a Fab′ fragment, a Fab₂ fragment, a F(ab′)2 fragment, a Fv fragment, a Fd′ fragment, and a Fd fragment.

In one aspect, said size exclusion chromatography system is coupled to said mass spectrometer.

In one aspect, a mobile phase for said size exclusion chromatography comprises ammonium acetate, ammonium bicarbonate, ammonium formate, or a combination thereof. In another aspect, a mobile phase for said size exclusion chromatography comprises from 100 mM to 200 mM ammonium acetate, or about 150 mM ammonium acetate.

In one aspect, said denaturing solution comprises acetonitrile, formic acid, or a combination thereof. In another aspect, said denaturing solution comprises from 40% to 80% acetonitrile. In a further aspect, said denaturing solution comprises from 1% to 10% formic acid. In an additional aspect, said denaturing solution comprises about 60% acetonitrile and about 4% formic acid.

In one aspect, said denaturing solution is contacted to said size exclusion chromatography eluate using a T-mixer. In another aspect, a flow from said size exclusion chromatography system is from 0.1 mL/minute to 0.5 mL/minute or about 0.2 mL/minute. In a further aspect, a flow of said denaturing solution is from 0.1 mL/minute to 0.5 mL/minute or about 0.2 mL/minute.

In one aspect, said mass spectrometer is an electrospray ionization mass spectrometer, nano-electrospray ionization mass spectrometer, or a triple quadrupole mass spectrometer.

In one aspect, a flow splitter is used to couple said size exclusion chromatography system with said mass spectrometer and a detector. In a specific aspect, said detector is an ultraviolet detector or a fluorescence detector. In another specific aspect, a flow from said size exclusion chromatography system is split into a low flow to said mass spectrometer and a high flow to said detector. In a more specific aspect, said low flow is less than 20 μL/minute or less than 10 μL/minute.

This disclosure also provides methods for identifying, quantifying, and/or characterizing at least one interacting protein in a sample that interacts with a protein of interest. In some exemplary embodiments, the methods can comprise (a) contacting a sample including at least one complex of at least one interacting protein and a protein of interest to a solid surface to form an immobilized complex; (b) eluting said immobilized complex to form an enriched complex; (c) subjecting said enriched complex to size exclusion chromatography under native conditions to form a size exclusion chromatography eluate; (d) contacting said size exclusion chromatography eluate to a denaturing solution to form a denatured eluate; and (e) subjecting said denatured eluate to mass spectrometry analysis to identify, quantify, and/or characterize said at least one interacting protein.

In one aspect, said at least one interacting protein comprises a serum protein. In another aspect, said interaction is a non-covalent interaction.

In one aspect, said sample is selected from a group consisting of cell culture fluid, harvested cell culture fluid, drug substance, drug product, a tissue sample, blood, serum, saliva, or urine. In a specific aspect, said sample is human serum or mouse serum.

In one aspect, said protein of interest is selected from a group consisting of a recombinant protein, a therapeutic protein, an antibody, a bispecific antibody, a trispecific antibody, a multispecific antibody, an antibody fragment, a fusion protein, a trap protein, a single-chain variable fragment, and combinations thereof.

In one aspect, said protein of interest is a therapeutic antibody.

In one aspect, said solid surface is selected from a group consisting of a microplate, resin, agarose beads, or magnetic beads.

In one aspect, said solid surface is adhered to an antibody that can specifically bind to said protein of interest. In a specific aspect, said adhering is mediated by biotin and avidin or streptavidin. In another specific aspect, said antibody is an anti-Fc antibody.

In one aspect, the methods can further comprise subjecting said immobilized protein of interest to at least one washing step to remove non-specifically bound components.

In one aspect, the methods can further comprise contacting said immobilized protein of interest to at least one digestive enzyme. In a specific aspect, said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease, LysC endoproteinase, endoproteinase AspN, endoproteinase GluC, outer membrane protein T, immunoglobulin-degrading enzyme of Streptococcus pyogenes (IdeS), thermolysin, papain, pronase, V8 protease, active fragments thereof, homologs thereof, variants thereof, and combinations thereof. In a more specific aspect, said at least one digestive enzyme comprises IdeS or a variant thereof.

In one aspect, eluting said immobilized protein of interest or fragment thereof comprises subjecting said solid surface to centrifugation.

In one aspect, said fragment is selected from the group consisting of a Fab fragment, a Fab′ fragment, a Fab₂ fragment, a F(ab′)2 fragment, a Fv fragment, a Fd′ fragment, and a Fd fragment.

In one aspect, said size exclusion chromatography system is coupled to said mass spectrometer.

In one aspect, a mobile phase for said size exclusion chromatography comprises ammonium acetate, ammonium bicarbonate, ammonium formate, or a combination thereof. In another aspect, a mobile phase for said size exclusion chromatography comprises from 100 mM to 200 mM ammonium acetate, or about 150 mM ammonium acetate.

In one aspect, said denaturing solution comprises acetonitrile, formic acid, or a combination thereof. In another aspect, said denaturing solution comprises from 40% to 80% acetonitrile. In a further aspect, said denaturing solution comprises from 1% to 10% formic acid. In an additional aspect, said denaturing solution comprises about 60% acetonitrile and about 4% formic acid.

In one aspect, said denaturing solution is contacted to said size exclusion chromatography eluate using a T-mixer. In another aspect, a flow from said size exclusion chromatography system is from 0.1 mL/minute to 0.5 mL/minute or about 0.2 mL/minute. In a further aspect, a flow of said denaturing solution is from 0.1 mL/minute to 0.5 mL/minute or about 0.2 mL/minute.

In one aspect, said mass spectrometer is an electrospray ionization mass spectrometer, nano-electrospray ionization mass spectrometer, or a triple quadrupole mass spectrometer.

In one aspect, a flow splitter is used to couple said size exclusion chromatography system with said mass spectrometer and a detector. In a specific aspect, said detector is an ultraviolet detector or a fluorescence detector. In another specific aspect, a flow from said size exclusion chromatography system is split into a low flow to said mass spectrometer and a high flow to said detector. In a more specific aspect, said low flow is less than 20 μL/minute or less than 10 μL/minute.

This disclosure further provides methods for identifying, quantifying, and/or characterizing at least one biotransformation product of a protein of interest. In some exemplary embodiments, the methods can comprise (a) contacting a sample including at least one biotransformation product of a protein of interest to a solid surface to form an immobilized biotransformation product; (b) eluting said immobilized biotransformation product or a fragment thereof to form an enriched biotransformation product or fragment thereof; (c) subjecting said enriched biotransformation product or fragment thereof to size exclusion chromatography under native conditions to form a size exclusion chromatography eluate; (d) contacting said size exclusion chromatography eluate to a denaturing solution to form a denatured eluate; and (e) subjecting said denatured eluate to mass spectrometry analysis to identify, quantify, and/or characterize said at least one biotransformation product.

In one aspect, said at least one biotransformation product comprises a post-translational modification, truncation, aggregate, fragment, degradation product, or combination thereof. In another aspect, said protein of interest comprises a linker and said at least one biotransformation product comprises a clipped form of said linker.

In one aspect, said sample is selected from a group consisting of cell culture fluid, harvested cell culture fluid, drug substance, drug product, a tissue sample, blood, serum, saliva, or urine. In a specific aspect, said sample is human serum or mouse serum.

In one aspect, said protein of interest is selected from a group consisting of a recombinant protein, a therapeutic protein, an antibody, a bispecific antibody, a trispecific antibody, a multispecific antibody, an antibody fragment, a fusion protein, a trap protein, a single-chain variable fragment, and combinations thereof.

In one aspect, said protein of interest is a therapeutic antibody.

In one aspect, said solid surface is selected from a group consisting of a microplate, resin, agarose beads, or magnetic beads.

In one aspect, said solid surface is adhered to an antibody that can specifically bind to said protein of interest. In a specific aspect, said adhering is mediated by biotin and avidin or streptavidin. In another specific aspect, said antibody is an anti-Fc antibody.

In one aspect, the methods can further comprise subjecting said immobilized protein of interest to at least one washing step to remove non-specifically bound components.

In one aspect, the methods can further comprise contacting said immobilized protein of interest to at least one digestive enzyme. In a specific aspect, said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease, LysC endoproteinase, endoproteinase AspN, endoproteinase GluC, outer membrane protein T, immunoglobulin-degrading enzyme of Streptococcus pyogenes (IdeS), thermolysin, papain, pronase, V8 protease, active fragments thereof, homologs thereof, variants thereof, and combinations thereof. In a more specific aspect, said at least one digestive enzyme comprises IdeS or a variant thereof.

In one aspect, eluting said immobilized protein of interest or fragment thereof comprises subjecting said solid surface to centrifugation.

In one aspect, said fragment is selected from the group consisting of a Fab fragment, a Fab′ fragment, a Fab₂ fragment, a F(ab′)2 fragment, a Fv fragment, a Fd′ fragment, and a Fd fragment.

In one aspect, said size exclusion chromatography system is coupled to said mass spectrometer.

In one aspect, a mobile phase for said size exclusion chromatography comprises ammonium acetate, ammonium bicarbonate, ammonium formate, or a combination thereof. In another aspect, a mobile phase for said size exclusion chromatography comprises from 100 mM to 200 mM ammonium acetate, or about 150 mM ammonium acetate.

In one aspect, said denaturing solution comprises acetonitrile, formic acid, or a combination thereof. In another aspect, said denaturing solution comprises from 40% to 80% acetonitrile. In a further aspect, said denaturing solution comprises from 1% to 10% formic acid. In an additional aspect, said denaturing solution comprises about 60% acetonitrile and about 4% formic acid.

In one aspect, said denaturing solution is contacted to said size exclusion chromatography eluate using a T-mixer. In another aspect, a flow from said size exclusion chromatography system is from 0.1 mL/minute to 0.5 mL/minute or about 0.2 mL/minute. In a further aspect, a flow of said denaturing solution is from 0.1 mL/minute to 0.5 mL/minute or about 0.2 mL/minute.

In one aspect, said mass spectrometer is an electrospray ionization mass spectrometer, nano-electrospray ionization mass spectrometer, or a triple quadrupole mass spectrometer.

In one aspect, a flow splitter is used to couple said size exclusion chromatography system with said mass spectrometer and a detector. In a specific aspect, said detector is an ultraviolet detector or a fluorescence detector. In another specific aspect, a flow from said size exclusion chromatography system is split into a low flow to said mass spectrometer and a high flow to said detector. In a more specific aspect, said low flow is less than 20 μL/minute or less than 10 μL/minute.

This disclosure additionally provides methods for producing a pharmacokinetic profile of a protein of interest. In some exemplary embodiments, the methods can comprise (a) quantifying a concentration of said protein of interest at a first time point after administration of said protein of interest to a subject by: (i) obtaining a sample from said subject including said protein of interest at a first time point after administration of said protein of interest to said subject; and (ii) quantifying said protein of interest according to the methods described above; and (b) repeating step (a) for at least one additional time point to produce a pharmacokinetic profile of said protein of interest.

In one aspect, said pharmacokinetic profile includes at least one biotransformation product of said protein of interest. In a specific aspect, said at least one biotransformation product comprises a post-translational modification, truncation, aggregate, fragment, degradation product, or combination thereof. In another specific aspect, said protein of interest comprises a linker and said at least one biotransformation product comprises a clipped form of said linker.

In one aspect, said pharmacokinetic profile includes at least one interacting protein that interacts with said protein of interest. In a specific aspect, said at least one interacting protein comprises a serum protein. In another specific aspect, said interaction is a non-covalent interaction.

In one aspect, said sample is selected from a group consisting of cell culture fluid, harvested cell culture fluid, drug substance, drug product, a tissue sample, blood, serum, saliva, or urine. In a specific aspect, said sample is human serum or mouse serum.

In one aspect, said protein of interest is selected from a group consisting of a recombinant protein, a therapeutic protein, an antibody, a bispecific antibody, a trispecific antibody, a multispecific antibody, an antibody fragment, a fusion protein, a trap protein, a single-chain variable fragment, and combinations thereof.

In one aspect, said protein of interest is a therapeutic antibody.

In one aspect, said solid surface is selected from a group consisting of a microplate, resin, agarose beads, or magnetic beads.

In one aspect, said solid surface is adhered to an antibody that can specifically bind to said protein of interest. In a specific aspect, said adhering is mediated by biotin and avidin or streptavidin. In another specific aspect, said antibody is an anti-Fc antibody.

In one aspect, the methods can further comprise subjecting said immobilized protein of interest to at least one washing step to remove non-specifically bound components.

In one aspect, the methods can further comprise contacting said immobilized protein of interest to at least one digestive enzyme. In a specific aspect, said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease, LysC endoproteinase, endoproteinase AspN, endoproteinase GluC, outer membrane protein T, immunoglobulin-degrading enzyme of Streptococcus pyogenes (IdeS), thermolysin, papain, pronase, V8 protease, active fragments thereof, homologs thereof, variants thereof, and combinations thereof. In a more specific aspect, said at least one digestive enzyme comprises IdeS or a variant thereof.

In one aspect, eluting said immobilized protein of interest or fragment thereof comprises subjecting said solid surface to centrifugation.

In one aspect, said fragment is selected from the group consisting of a Fab fragment, a Fab′ fragment, a Fab₂ fragment, a F(ab′)2 fragment, a Fv fragment, a Fd′ fragment, and a Fd fragment.

In one aspect, said size exclusion chromatography system is coupled to said mass spectrometer.

In one aspect, a mobile phase for said size exclusion chromatography comprises ammonium acetate, ammonium bicarbonate, ammonium formate, or a combination thereof. In another aspect, a mobile phase for said size exclusion chromatography comprises from 100 mM to 200 mM ammonium acetate, or about 150 mM ammonium acetate.

In one aspect, said denaturing solution comprises acetonitrile, formic acid, or a combination thereof. In another aspect, said denaturing solution comprises from 40% to 80% acetonitrile. In a further aspect, said denaturing solution comprises from 1% to 10% formic acid. In an additional aspect, said denaturing solution comprises about 60% acetonitrile and about 4% formic acid.

In one aspect, said denaturing solution is contacted to said size exclusion chromatography eluate using a T-mixer. In another aspect, a flow from said size exclusion chromatography system is from 0.1 mL/minute to 0.5 mL/minute or about 0.2 mL/minute. In a further aspect, a flow of said denaturing solution is from 0.1 mL/minute to 0.5 mL/minute or about 0.2 mL/minute.

In one aspect, said mass spectrometer is an electrospray ionization mass spectrometer, nano-electrospray ionization mass spectrometer, or a triple quadrupole mass spectrometer.

In one aspect, a flow splitter is used to couple said size exclusion chromatography system with said mass spectrometer and a detector. In a specific aspect, said detector is an ultraviolet detector or a fluorescence detector. In another specific aspect, a flow from said size exclusion chromatography system is split into a low flow to said mass spectrometer and a high flow to said detector. In a more specific aspect, said low flow is less than 20 μL/minute or less than 10 μL/minute.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 illustrates a workflow of the SCX-MS method of the present invention according to an exemplary embodiment.

FIG. 2 shows a comparison of the performance of different SCX columns in SCX-MS total ion chromatograms (TICs) for the separation of antibodies according to an exemplary embodiment.

FIG. 3A shows SCX-MS TICs for a range of different antibodies according to an exemplary embodiment.

FIG. 3B shows mass spectra of mAb1 at varying concentrations according to an exemplary embodiment.

FIG. 3C shows mass spectra of mAb2 at varying concentrations according to an exemplary embodiment.

FIG. 4A shows a SCX-MS TIC of mAb1 Fab₂ and internal standard mAb2 Fab₂ according to an exemplary embodiment.

FIG. 4B shows a linearity of measured mAb1 concentration between 20 pg and 20 ng in neat solution compared to an internal standard according to an exemplary embodiment.

FIG. 4C shows a linearity of measured mAb1 concentration between 20 ng and 2 μg in neat solution compared to an internal standard according to an exemplary embodiment.

FIG. 4D shows mass spectra for mAb1 at concentrations between 20 pg and 2 μg in neat solution according to an exemplary embodiment.

FIG. 5A shows a linearity of measured mAb1 concentration in serum when normalized to an internal standard according to an exemplary embodiment.

FIG. 5B shows an inset from FIG. 5A illustrating a linearity of measured mAb1 concentration at low concentrations in serum according to an exemplary embodiment.

FIG. 5C shows an inset from FIG. 5B illustrating a linearity of measured mAb1 concentration at low concentrations in serum according to an exemplary embodiment.

FIG. 6A shows a linearity of measured mAb1 concentration in serum without normalization to an internal standard according to an exemplary embodiment.

FIG. 6B shows an inset from FIG. 6A illustrating a linearity of measured mAb1 concentration at low concentrations in serum according to an exemplary embodiment.

FIG. 6C shows an inset from FIG. 6B illustrating a linearity of measured mAb1 concentration at low concentrations in serum according to an exemplary embodiment.

FIG. 7A shows a limit of detection (LOD) of mAb1 in serum in a mass spectrum according to an exemplary embodiment.

FIG. 7B shows a limit of quantitation (LOQ) of mAb1 in serum in a mass spectrum according to an exemplary embodiment.

FIG. 8 illustrates a workflow of the SEC-PCD-MS method of the present invention, according to an exemplary aspect.

FIG. 9 shows separation and identification of a serum protein and drug from a drug-serum protein complex using the SEC-PCD-MS method of the present invention, according to an exemplary aspect.

FIG. 10 shows a change in proteoforms of an antibody drug as a function of time after administration to a subject, according to an exemplary aspect.

FIG. 11 shows an identification of biotransformation products using the SEC-PCD-MS method of the present invention, according to an exemplary aspect.

FIG. 12 shows a pharmacokinetic profile of proteoforms of an antibody drug determined using the SEC-PCD-MS method of the present invention, according to an exemplary aspect.

### DETAILED DESCRIPTION

Therapeutic peptides or proteins can become heterogeneous due to various post-translational modifications (PTMs), protein degradation, enzymatic modifications, and chemical modifications, which can be introduced at any point during and after the production and purification of peptides or proteins. Multispecific monoclonal antibodies (mAbs) present additional complexity due to the presence of a linker, for example a G4S linker (a sequence of four glycines and a serine), to an additional Fab arm, which may undergo additional modifications including linker clipping.

Identification and characterization of the heterogeneous variants are critical to controlling the quality attributes of the biophysical characteristics of biopharmaceutical products. There are needs in the biopharmaceutical industry for rapid sensitive high-throughput analytical methods to control and monitor the production and purification of therapeutic peptides or proteins, such as the production of monoclonal antibodies or antibody-drug conjugates.

Processing of a therapeutic peptide or protein in vivo after administration further determines features such as the efficacy and safety of the therapeutic. Properties such as the pharmacokinetics (PK) and pharmacodynamics (PD) of a peptide or protein may only become apparent after administration. Additionally, modifications to a therapeutic peptide or protein may continue to be made in vivo, resulting in biotransformation products that may not be predictable during manufacturing. Furthermore, physiological molecules such as serum proteins may interact with the therapeutic peptide or protein after administration, affecting the in vivo properties of the therapeutic. Thus, in order to fully understand important attributes of a therapeutic, biological samples may be analyzed, which present increased complexity and challenges to sensitive and specific characterization and quantification of a protein or peptide of interest.

Electrospray ionization mass spectrometry (ESI MS)-based intact protein analysis has become an essential tool for the characterization of therapeutic proteins during development. Most commonly, MS is coupled with reversed phase liquid chromatography (RPLC) under denaturing conditions. However, the sensitivity of this method, and the signal-to-noise ratio produced by the resulting complex sample with a wide range of analyte charge states, has limits which may make it unreliable for accurate quantitation of low-abundance antibodies.

Recently, LC-MS systems comprising native ion exchange chromatography coupled online to ESI MS have been described (Yan et al., 2020, J Am Soc Mass Spectrom, 31:2171-2179). The use of native strong cation exchange chromatography (SCX)-MS provides a number of advantages for analysis of therapeutic antibodies compared to conventional denaturing RPLC-MS. Native SCX-MS may demonstrate high sensitivity and a wide dynamic range compared to RPLC, and a superior ability to separate a target analyte from matrix, such as for example serum proteins in a serum sample. A native SCX-MS profile may also feature superior MS spatial resolution, making it easier to detect protein variants or biotransformation products.

Additionally, LC-MS systems comprising native size exclusion chromatography coupled online to ESI MS, with postcolumn denaturation prior to ionization, have been described (Yan et al., 2021, J Am Soc Mass Spectrom, 32:2885-2894). Using MS-compatible mobile phases that can preserve protein conformation and non-covalent interactions, native SEC-MS (nSEC-MS) can provide rapid and improved separation of analytes and identification of size variants and biotransformation products based on accurate mass measurement. In addition, thanks to the recent advances in both methodology and instrumentation, nSEC-MS has become a highly sensitive method that can readily detect very low levels of variants (e.g., at 0.01%) directly from complex samples.

Despite these notable successes, application of the nSEC-MS method alone still cannot obtain a complete profile of protein size variants. First, as a non-denaturing method, nSEC-MS analysis does not distinguish between non-covalently and covalently bound complexes, unless clear mass differences resulting from the covalent crosslinks can be detected. Unfortunately, the latter can be extremely difficult to achieve, due to both insufficient chromatographical resolution and mass resolving power for large complexes. For instance, dimer species formed by different mechanisms (e.g., non-covalent and covalent interactions) are often co-eluted during SEC separation and measured with an averaged mass by MS detection. Therefore, the distribution of non-covalent and covalent dimer species cannot be directly determined by an nSEC-MS method. Second, compared to well-expected oligomeric species (e.g., dimer, trimer, tetramer, etc.), confident identification of unconventional complexes (e.g., mAb monomer complexed with additional light chains, or variants of a multispecific mAb monomer) often cannot be established by intact mass measurement alone. This is because reduced mass accuracy is often expected for mass measurement of large protein species present at low abundances, which can lead to ambiguous mass assignments.

To overcome these challenges, a post-column denaturation-assisted nSEC-MS method (nSEC-PCD-MS) can be used to dissociate SEC-resolved, non-covalently bound protein complexes (for example, a therapeutic protein interacting with a serum protein) into constituent components for subsequent MS detection. As a result, this approach enables simultaneous detection of both non-covalent and non-dissociable complexes under identical SEC separation conditions. In addition, this strategy improves the identification of heterogeneous species by 1) confirming the identities of constituent subunits dissociated from non-covalent complexes; and 2) achieving more accurate mass measurement of non-dissociable species by removing interference from co-eluting, non-covalent species.

As described above, there exists a need for sensitive methods to characterize and quantitate therapeutic proteins and peptides, such as therapeutic antibodies and multispecific antibodies, in a sample. This disclosure sets forth novel native SCX-MS methods and native SEC-PCD-MS methods for characterizing an antibody, suitable for development of therapeutic antibodies, including multispecific antibodies. The methods can be used to identify, quantify, and characterize a protein of interest, biotransformation products of the protein of interest, and interacting proteins that interact with the protein of interest, including to produce pharmacokinetic profiles after administration of the protein of interest to a subject.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, a therapeutic protein, an antibody, a bispecific antibody, a trispecific antibody, a multispecific antibody, an antibody fragment, a monoclonal antibody, a fusion protein, a trap protein, a single-chain variable fragment (scFv), and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1 CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 (or “Fab₂”) fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. In some exemplary embodiments, an antibody fragment may be produced by digestion with the digestive enzyme IdeS or a variant thereof. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22343 filed Jul. 29, 2016; and U.S. Ser. No. 15/814,095, filed Nov. 15, 2017.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein. In some aspects, a multispecific antibody includes a linker between a first antibody, antibody fragment, antibody fusion protein, antibody-derived protein, or other antigen-binding protein and a second antibody, antibody fragment, antibody-derived protein, antibody fusion protein, or other antigen-binding protein. In some aspects, the linker is a G45 linker. In some aspects, the multispecific antibody comprises a monospecific antibody, a bispecific antibody, or a trispecific antibody with a linker to a single-chain variable fragment (scFv) having an additional binding specificity.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “Fc fusion proteins” as used herein includes part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. Sci. USA 88: 10535, 1991; Byrn et al., Nature 344:677, 1990; and Hollenbaugh et al., “Construction of Immunoglobulin Fusion Proteins”, in Current Protocols in Immunology, Suppl. 4, pages 10.19.1-10.19.11, 1992. “Receptor Fc fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). For example, an Fc-fusion protein may be a trap, such as, for example, an IL-1 trap (e.g., Rilonacept, which contains the IL-1 RAcP ligand binding region fused to the IL-1R1 extracellular region fused to Fc of hIgG1; see U.S. Pat. No. 6,927,004, which is herein incorporated by reference in its entirety), or a VEGF Trap (e.g., Aflibercept, which contains the Ig domain 2 of the VEGF receptor Flt1 fused to the Ig domain 3 of the VEGF receptor Flk1 fused to Fc of hIgG1; e.g., SEQ ID NO:1; see U.S. Pat. Nos. 7,087,411 and 7,279,159, which are herein incorporated by reference in their entirety).

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

As used herein, “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling.

A sample may also be taken from a subject prior to and/or after administration of a therapeutic peptide or protein, in which case it may be a “biological sample” or “PK sample.” A biological sample may be, for example, a tissue sample, a blood sample, a serum sample, a saliva sample, or a urinary sample. In an exemplary embodiment, a serum sample is taken from a subject in order to characterize and/or quantify a protein of interest, a biotransformation product of a protein of interest, and/or interacting proteins that interact with a protein of interest after administration of the protein of interest to a subject. In some aspects, a biological sample is taken from a mouse. In some aspects, the subject is an animal, for example a non-human animal or a human. In some aspects, the subject is a mouse.

A sample may comprise components with known or unknown interactions with a protein of interest. For example, a therapeutic protein administered to a subject may interact with a serum protein (an “interacting protein”), which may affect the safety, efficacy, pharmacokinetics or pharmacodynamics of the therapeutic protein. This disclosure provides methods for identifying, quantifying, and characterizing an interacting protein that interacts with a protein of interest, and for producing a pharmacokinetic profile to determine how this interaction changes as a function of time after administration of a protein of interest to a subject. In particular, an interacting protein may form a non-covalent complex with a protein of interest that is maintained when a protein of interest is enriched, for example using immunoprecipitation. This complex may also be maintained during a chromatographic processing step in native conditions, for example SEC, allowing for a separation between interacting and non-interacting forms of the respective proteins. The complex may then be dissociated using postcolumn denaturation, allowing for sensitive detection using mass spectrometry and analysis of the interacting protein.

As used herein, the term “impurity” can include any undesirable protein present in the protein biopharmaceutical product. Impurity can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables. Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S-S linked, oxidized, or altered conjugated forms (e.g., glycosylation, phosphorylation). Modified forms can also include any post-translational modification form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

As used herein, the general term “post-translational modifications” or “PTMs” refer to covalent modifications that polypeptides undergo, either during (co-translational modification) or after (post-translational modification) their ribosomal synthesis. PTMs are generally introduced by specific enzymes or enzyme pathways. Many occur at the site of a specific characteristic protein sequence (signature sequence) within the protein backbone. Several hundred PTMs have been recorded, and these modifications invariably influence some aspect of a protein's structure or function (Walsh, G. “Proteins” (2014) second edition, published by Wiley and Sons, Ltd., ISBN: 9780470669853). The various post-translational modifications include, but are not limited to, cleavage, N-terminal extensions, protein degradation, acylation of the N-terminus, biotinylation (acylation of lysine residues with a biotin), amidation of the C-terminal, glycosylation, iodination, covalent attachment of prosthetic groups, acetylation (the addition of an acetyl group, usually at the N-terminus of the protein), alkylation (the addition of an alkyl group (e.g. methyl, ethyl, propyl) usually at lysine or arginine residues), methylation, adenylation, ADP-ribosylation, covalent cross links within, or between, polypeptide chains, sulfonation, prenylation, Vitamin C dependent modifications (proline and lysine hydroxylations and carboxy terminal amidation), Vitamin K dependent modification wherein Vitamin K is a cofactor in the carboxylation of glutamic acid residues resulting in the formation of a γ-carboxyglutamate (a glu residue), glutamylation (covalent linkage of glutamic acid residues), glycylation (covalent linkage glycine residues), glycosylation (addition of a glycosyl group to either asparagine, hydroxylysine, serine, or threonine, resulting in a glycoprotein), isoprenylation (addition of an isoprenoid group such as farnesol and geranylgeraniol), lipoylation (attachment of a lipoate functionality), phosphopantetheinylation (addition of a 4′-phosphopantetheinyl moiety from coenzyme A, as in fatty acid, polyketide, non-ribosomal peptide and leucine biosynthesis), phosphorylation (addition of a phosphate group, usually to serine, tyrosine, threonine or histidine), and sulfation (addition of a sulfate group, usually to a tyrosine residue). The post-translational modifications that change the chemical nature of amino acids include, but are not limited to, citrullination (the conversion of arginine to citrulline by deimination), and deamidation (the conversion of glutamine to glutamic acid or asparagine to aspartic acid). The post-translational modifications that involve structural changes include, but are not limited to, formation of disulfide bridges (covalent linkage of two cysteine amino acids) and proteolytic cleavage (cleavage of a protein at a peptide bond). Certain post-translational modifications involve the addition of other proteins or peptides, such as ISGylation (covalent linkage to the ISG15 protein (Interferon-Stimulated Gene)), SUMOylation (covalent linkage to the SUMO protein (Small Ubiquitin-related MOdifier)) and ubiquitination (covalent linkage to the protein ubiquitin). See European Bioinformatics Institute Protein Information ResourceSlB Swiss Institute of Bioinformatics, European Bioinformatics Institute Drs—Drosomycin precursor—Drosophila melanogaster (Fruit fly)—Drs gene & protein, uniprot.org/docs/ptmlist (last visited Jan. 15, 2019) for a more detailed controlled vocabulary of PTMs curated by UniProt.

Post-translational modifications, charge variants, or size variants of a therapeutic peptide or protein may arise at any point during the production, manufacture, storage, delivery, or administration of a therapeutic peptide or protein. Additional modifications to a peptide or protein may occur in vivo after administration to a subject, in a process referred to as “biotransformation.” Biotransformation products may have modified properties compared to a pre-administration therapeutic. Biotransformation often leads to a reduction in size of a therapeutic, such that detection methods with higher sensitivity for smaller analytes may be preferred. In some exemplary embodiments, the method of the present invention features high sensitivity for biotransformation products of a protein of interest.

In some exemplary embodiments, the method for characterizing and/or quantifying a protein of interest can optionally comprise enriching a protein of interest in the sample matrix by contacting the sample to a solid surface to immobilize the protein of interest. The protein of interest or a fragment thereof may then be eluted to form an enriched protein of interest. In some aspects, the solid surface may also immobilize biotransformation products of the protein of interest. In some aspects, a complex including the protein of interest and at least one interacting protein may be immobilized by the solid surface.

For example, a protein of interest (and/or a biotransformation product or an interacting protein thereof) may be enriched using immunoprecipitation (IP). As used herein, the term “immunoprecipitation” can include a process of precipitating a protein antigen out of solution using an antibody that specifically binds to that particular protein. Immunoprecipitation may be direct, in which antibodies for the target protein are immobilized on a solid-phase substrate (solid surface), or indirect, in which free antibodies are added to the protein mixture and later captured with, for example, protein A/G beads.

In some exemplary embodiments, the solid surface may be a microplate, resin, or beads, for example agarose beads or magnetic beads. Beads may be coated in streptavidin in order to facilitate adherence to an antibody. A biotinylated “capture” antibody may then be contacted to the streptavidin-coated beads, adhering to the beads and forming “immunoprecipitation beads” capable of binding to the antigen of the adhered antibody. In some exemplary embodiments, the adhered capture antibody may be an anti-Fc antibody, and may specifically be an anti-human Fc antibody.

An anti-human Fc antibody will preferentially bind to the Fc domain of any human antibody, such as, for example, a therapeutic antibody, and thus may be used to immunoprecipitate or “pull down” a therapeutic antibody from a sample, allowing it to be enriched for analysis. After immunoprecipitation of a therapeutic antibody, a digestive enzyme may be contacted to the immunoprecipitation mixture to cleave the therapeutic antibody and release antibody fragments that may then be eluted for further analysis. In an exemplary embodiment, IdeS or variants thereof are used as a digestive enzyme. IdeS cleavage produces two antibody fragments: an Fc fragment and a Fab₂ fragment. When the Fc domain of a therapeutic antibody is bound to an anti-human Fc capture antibody, cleavage with IdeS will result in the release of an unbound Fab₂ fragment, which can then be eluted for further analysis. In an exemplary embodiment, eluted Fab₂ fragments are subjected to liquid chromatography-mass spectrometry analysis, in particular native SCX-MS or native SEC-MS.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary embodiments, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Additionally, IdeS digestion has a high efficiency, allowing for high recovery of an analyte. The digestion and elution process may be performed under native conditions, allowing for simple coupling to a native LC-MS system.

IdeS or variants thereof are commercially available and may be marketed as, for example, FabRICATOR® or FabRICATOR Z®.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography.

In some exemplary embodiments, the method for characterizing and/or quantifying a protein of interest can include the use of strong cation exchange (SCX) chromatography. Cation exchange chromatography is a subset of ion exchange chromatography that uses a stationary phase presenting a negatively charged functional group in order to capture positively charged analytes. The pH of the chromatography buffer can be gradually adjusted in order to release and elute the analytes in order of pI.

Cation exchange chromatography uses a “cation exchange chromatography material.” Cation exchange chromatography can be further subdivided into, for example, strong cation exchange (SCX) or weak cation exchange, depending on the cation exchange chromatography material employed. Cation exchange chromatography materials with a sulfonic acid group (S) may be used in strong cation exchangers, while cation exchange chromatography materials with a carboxymethyl group (CM) may be used in weak cation exchangers. Strong cation exchangers include, for example SOURCE S, which uses a functional group of methyl sulfate, and SP Sepharose, which uses a functional group of sulfopropyl. Weak cation exchangers include, for example, CM-Cellulose, which uses a functional group of carboxymethyl. SCX may be preferred because a wider range of pH buffers may be used without losing the charge of the strong cation exchanger, allowing for effective separation of analytes with a wide pI range.

Cation exchange chromatography materials are available under different names from a multitude of companies such as, for example, Bio-Rex, Macro-Prep CM (available from BioRad Laboratories, Hercules, Calif., USA), weak cation exchanger WCX 2 (available from Ciphergen, Fremont, Calif, USA), Dowex MAC-3 (available from Dow chemical company, Midland, Mich., USA), Mustang C (available from Pall Corporation, East Hills, N.Y., USA), Cellulose CM-23, CM-32, CM-52, hyper-D, and partisphere (available from Whatman plc, Brentford, UK), Amberlite IRC 76, IRC 747, IRC 748, GT 73 (available from Tosoh Bioscience GmbH, Stuttgart, Germany), CM 1500, CM 3000 (available from BioChrom Labs, Terre Haute, Ind., USA), and CM-Sepharose Fast Flow (available from GE Healthcare, Life Sciences, Germany). In addition, commercially available cation exchange resins further include carboxymethyl-cellulose, Bakerbond ABX, sulphopropyl (SP) immobilized on agarose (e.g. SP-Sepharose Fast Flow or SP-Sepharose High Performance, available from GE Healthcare—Amersham Biosciences Europe GmbH, Freiburg, Germany) and sulphonyl immobilized on agarose (e.g. S-Sepharose Fast Flow available from GE Healthcare, Life Sciences, Germany).

Cation exchange chromatography materials include mixed-mode chromatography materials performing a combination of ion exchange and hydrophobic interaction technologies (e.g., Capto adhere, Capto MMC, MEP HyperCell, Eshmuno HCX, etc.), mixed-mode chromatography materials performing a combination of anion exchange and cation exchange technologies (e.g., hydroxyapatite, ceramic hydroxyapatite, etc.), and the like. Cation exchange chromatography materials that may be used in cation exchange chromatography in the present invention may include, but are not limited to, all the commercially available cation exchange chromatography materials as described above.

While denaturing RPLC-MS is a conventional technique in the characterization of therapeutic proteins, native SCX-MS may provide analytical advantages as described herein. For example, native SCX-MS may provide improved sensitivity and specificity of detection. In cases where the detection limits of RPLC and SCX are comparable, SCX may provide superior data quality and a higher signal-to-noise ratio. SCX may have an improved ability to separate a target analyte from matrix proteins, for example serum proteins in a serum sample, and additionally may have an improved ability to separate biotransformation products of a protein of interest. Thus, the preferred chromatography for the method of the present invention is native SCX, and disclosed herein is a novel method of characterizing and/or quantifying a protein of interest using native SCX.

In some aspects, the methods for characterizing and/or quantifying a protein of interest can include the use of size exclusion chromatography (SEC). Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename. “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

In some aspects, a mobile phase for SEC is selected to be compatible with online coupling to a mass spectrometer. In some aspects, the mobile phase comprises ammonium acetate, ammonium bicarbonate, ammonium formate, or a combination thereof. In some aspects, the mobile phase comprises a concentration of ammonium acetate from 100 mM to 200 mM, from 120 mM to 180 mM, from 140 mM to 160 mM, from 145 mM to 155 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work using nanoelectrospray or nanospray.

The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, SCX-MS and/or SEC-MS can be performed under native conditions.

As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In exemplary embodiments, native MS allows for better spatial resolution compared to non-native MS, improving detection of biotransformation products of a therapeutic protein. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE1176-1192 (2015).

In some aspects, SCX-MS and/or SEC-MS can be performed under non-native conditions. A peptide or protein of interest may be prepared by, for example, alkylation, reduction, denaturation, and/or digestion. In other aspects, SCX and/or SEC may be performed under native conditions and subsequent MS may be performed under near-native conditions, with the LC eluate being denatured by a denaturating solution before being contacted to the MS.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, a “denaturing solution” can refer to a solution comprising at least one denaturing agent. A denaturing agent can include any of those listed above, and in particular can include, for example, acetonitrile, formic acid, or a combination thereof. In some aspects, a percent of acetonitrile in a denaturing solution can be from 40% to 80%, from 50% to 70%, from 55% to 65%, from 59% to 61%, about 40%, about 45%, about 50%, about 55%, about 59%, about 60%, about 61%, about 65%, about 70%, about 75%, or about 80%. In some aspects, a percent of formic acid in a denaturing solution can be from 1% to 10%, from 2% to 8%, from 2% to 6%, from 3% to 5%, from 3.5% to 4.5%, about 1%, about 2%, about 3%, about 3.5%, about 4%, about 4.5%, about 5%, about 6%, about 7%, about 8%, about 9%, or about 10%. In some aspects, a denaturing solution may comprise from 50% to 70% acetonitrile and from 2% to 8% formic acid. In some aspects, a denaturing solution may comprise about 60% acetonitrile and about 4% formic acid.

A denaturing solution can be introduced into the flow of eluate from a chromatography system prior to the flow entering a mass spectrometer to form a denatured eluate, a process called postcolumn denaturation (PCD). The denaturing solution can be introduced using a mixer, for example a T-mixer. In some aspects, the flow from the chromatography system can be about the same as the flow of denaturing solution. In some aspects, a flow from a chromatography system can be from 0.1 mL/minute to 0.5 mL/minute, about 0.1 mL/minute, about 0.2 mL/minute, about 0.3 mL/minute, about 0.4 mL/minute, or about 0.5 mL/minute. In some aspects, a flow of denaturing solution can be from 0.1 mL/minute to 0.5 mL/minute, about 0.1 mL/minute, about 0.2 mL/minute, about 0.3 mL/minute, about 0.4 mL/minute, or about 0.5 mL/minute.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

In some aspects, a flow from a chromatography system (before, after, or excluding a flow of a denaturing solution) may be split by a flow splitter prior to entering a mass spectrometer, for example to reduce a total volume of solution entering a mass spectrometer or to reduce an amount of salt entering a mass spectrometer. In some aspects, a first flow may be directed to a mass spectrometer and a second flow may be directed to a detector, for example an ultraviolet (UV) detector or a fluorescence detector. In some aspects, the flow from a chromatography system is split into a low flow to the mass spectrometer and a high flow to the detector. In some aspects, the low flow is less than 20 μL/minute, less than 15 μL/minute, less than 10 μL/minute, or less than 5 μL/minute.

In some exemplary aspects, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information, or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post-translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com//proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsfedu/prospector/mshome.htm), Byonic (proteinmetrics.com/products/byonic) or Sequest (fields.scripps.edu/sequest).

In some exemplary embodiments, the mass spectrometer is coupled to the chromatography system, for example, SCX or SEC.

In some exemplary embodiments, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some aspects, the mass spectrometer in the method or system of the present application can be an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-MRM-MS (liquid chromatography-multiple reaction monitoring-mass spectrometry) analyses.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

It is understood that the present invention is not limited to any of the aforesaid protein(s) of interest, antibody(s), antibody fragment(s), sample(s), impurity(s), PTM(s), immunoprecipitation method(s), liquid chromatography method(s) or system(s), mass spectrometer(s), alkylating agent(s), reducing agent(s), digestive enzyme(s), database(s), or bioinformatics tool(s), and any protein(s) of interest, antibody(s), antibody fragment(s), sample(s), impurity(s), PTM(s), immunoprecipitation method(s), liquid chromatography method(s) or system(s), mass spectrometer(s), alkylating agent(s), reducing agent(s), digestive enzyme(s), database(s), or bioinformatics tool(s) can be selected by any suitable means.

The present invention will be more fully understood by reference to the following Examples. They should not, however, be construed as limiting the scope of the invention.

### EXAMPLES

### Example 1. Materials and Methods for Native SCX-MS Analysis

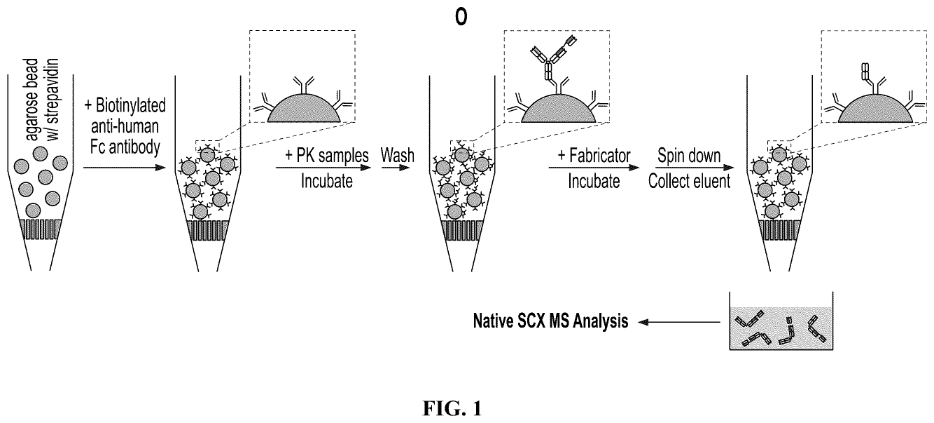

An exemplary embodiment of the method of the present invention is illustrated in FIG. 1. The first component shown is a cartridge containing agarose beads conjugated with streptavidin moieties (a solid surface, or solid-phase substrate). Biotinylated anti-human Fc antibody is then added to the cartridge and bound to the streptavidin beads to produce immunoprecipitation beads. Biotinylated anti-human Fc may be produced or commercially purchased. An exemplary biotin-streptavidin reaction comprises incubation at about room temperature for about 15 minutes. Samples including the analyte are then added to the cartridge and incubated to immunoprecipitate or “pull down” the analyte, forming an immobilized protein. An exemplary immunoprecipitation process comprises incubation at about room temperature for about 1 hour. The example illustrated is a sample from a pharmacokinetic study comprising a trispecific antibody as the protein of interest and analyte, but the method of the present invention is not limited to this example and may be applied to any appropriate sample comprising any antibody or antibody-related protein.

The sample is then washed to remove non-specifically bound components. An exemplary washing step comprises washing the cartridge with 6 cartridge volumes of HBS-EP buffer (Cytiva), followed by 6 cartridge volumes of Tris-HCl (10 mM, pH 7.5). A digestive enzyme, for example IdeS or a variant thereof, is then added to the cartridge and incubated, which leads to cleavage of the bound analyte, for example separating the Fc fragment from the Fab₂ fragment of an antibody. An exemplary digestion step comprises adding 40 units of the IdeS protein FabRICATOR® (Genovis), or 1 unit of digestive enzyme per μg of analyte, and incubating at about 37° C. for about 30 minutes to about 1 hour. The cartridge is centrifuged (“spun down”) to elute freed Fab₂ fragments, and the eluate is collected for subsequent analysis, for example native SCX-MS analysis.

Exemplary methods for native SCX-MS analysis are described in Yan et al., 2020, J Am Soc Mass Spectrom, 31:2171-2179, which is hereby incorporated by reference. In an exemplary embodiment, SCX-MS conditions are as follows. The SCX column is YMC BioPro IEX SF 4.6×50 mm, 5 μm. The column temperature is 45° C. Mobile phase A (MPA) comprises 10 mM ammonium acetate, and mobile phase B (MPB) comprises 300 mM ammonium acetate. The flow rate is 0.4 mL/minute. The gradient is: 0-1 minutes: 100% MPA; 1-9 minutes: 100% MPA to 100% MPB; 9-10.5 minutes: 100% MPB; 10.5-10.6 minutes: 100% MPB to 100% MPA; and 10.6-15 minutes: 100% MPA.

The MS resolution is set at 12,500 (UHMIR). The capillary spray voltage is set at 3.0 kV. The capillary temperature is set at 350° C. The S-lens RF level is set at 200. The in-source fragmentation energy is set at 100. The HCD trapping gas pressure is set at 3. Mass spectra are acquired with an m/z range window between 2000 and 15,000.

### Example 2. Selection of SCX Column

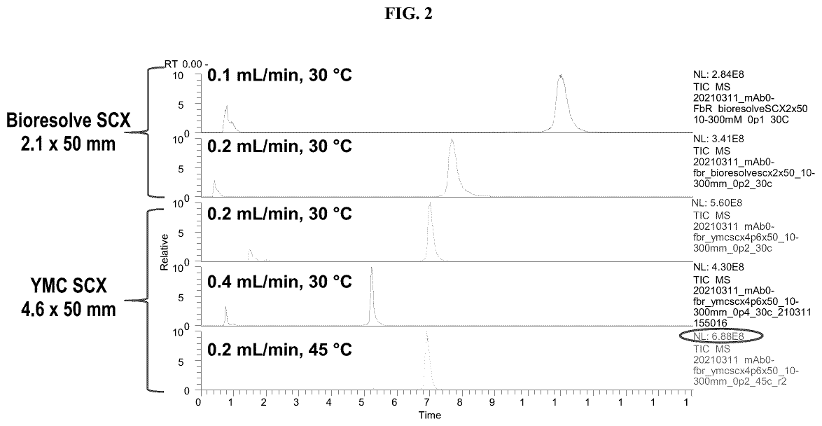

The performance of multiple SCX columns was compared to optimize the method of the present invention. Fab₂ fragments were prepared as described above and subjected to native SCX-MS analysis. Bioresolve SCX 2.1×50 mm was compared to YMC SCX 4.6×50 mm. SCX-MS total ion chromatograms (TICs) for each column are shown in FIG. 2, with corresponding flow rates and temperature for each experiment shown. Based on the demonstrated sensitivity of the method, YMC SCX 4.6×50 mm was used for further experiments, using an 8 minute gradient of 10 to 300 mM ammonium acetate buffer.

### Example 3. Establishing Limit of Detection and Limit of Quantitation in Neat Solution

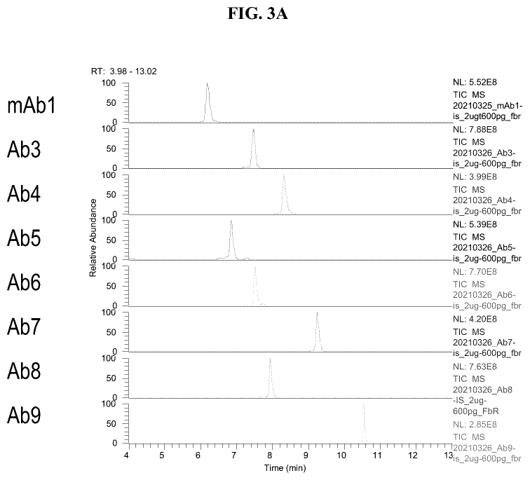

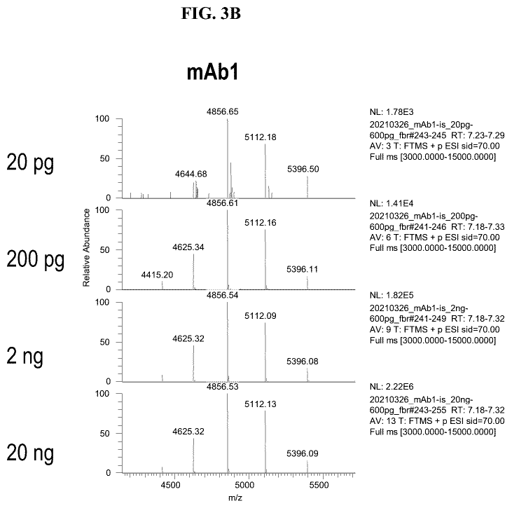

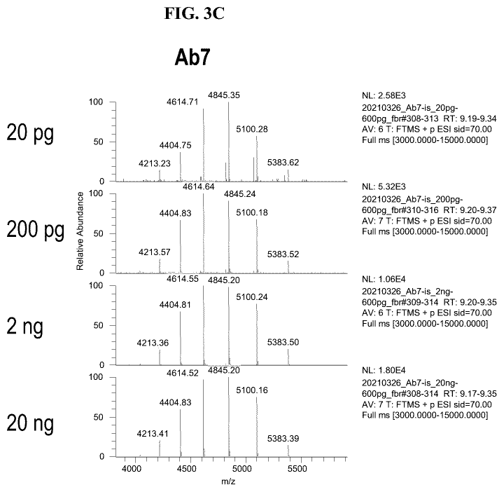

The native SCX-MS method of the present invention was tested on Fab₂ fragments in neat solution to establish a limit of detection (LOD). Neat solution comprised an antibody analyte and an internal standard antibody (300 pg/μL, or 600 pg on the column) in 10 mM Tris-HCl buffer (pH 7.5). A range of antibodies was tested as the analyte, with pI ranging from high to low, as shown in FIG. 3A. pI ranges of tested antibodies were between 6.28 and 8.15. Sample amounts tested ranged from 20 pg to 2 μg on the column, with concentration ranges between 10 pg/μL and 1 μg/μL.

A 15 minute SCX run was performed for each sample, each with a 0.2 mL/minute flow rate, except for Ab9. Antibodies tested included IgG1 and IgG4 antibodies, and mAbs and bsAbs, representing a diverse variety of therapeutic antibodies. The method of the present invention was capable of effectively separating and analyzing each antibody with high sensitivity. Mass spectra from two exemplary antibodies at a range of concentrations between 20 pg and 20 ng are shown in FIGS. 3B and 3C. The absolute LOD of Fab₂ fragments using the method of the present invention under these conditions was determined to be 20 pg.

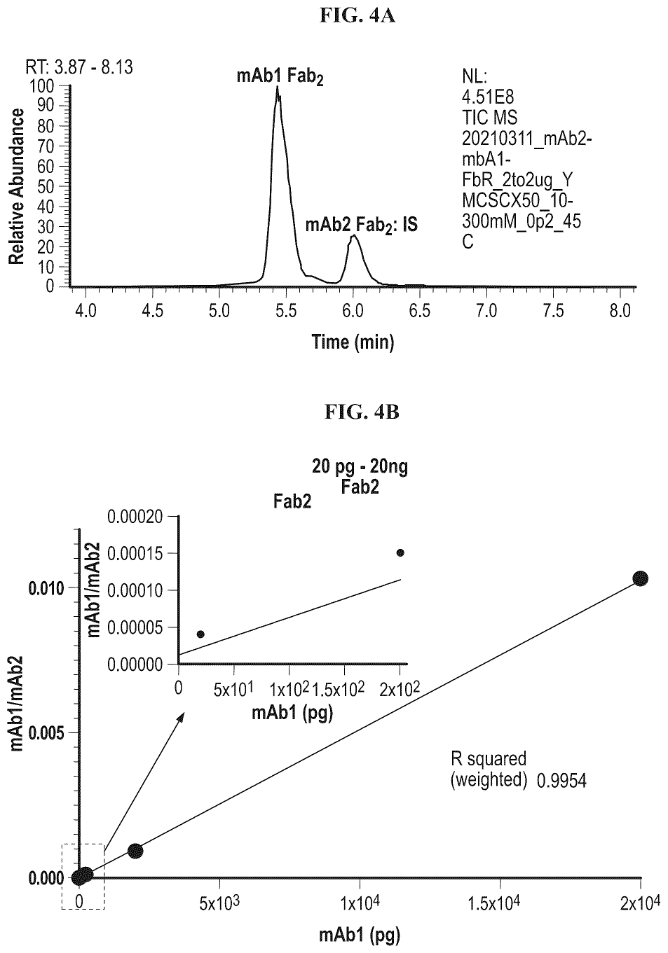

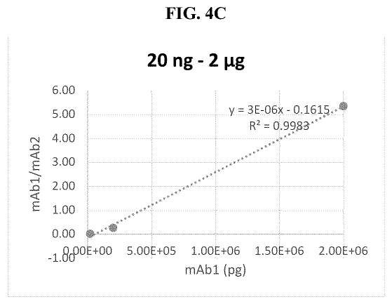

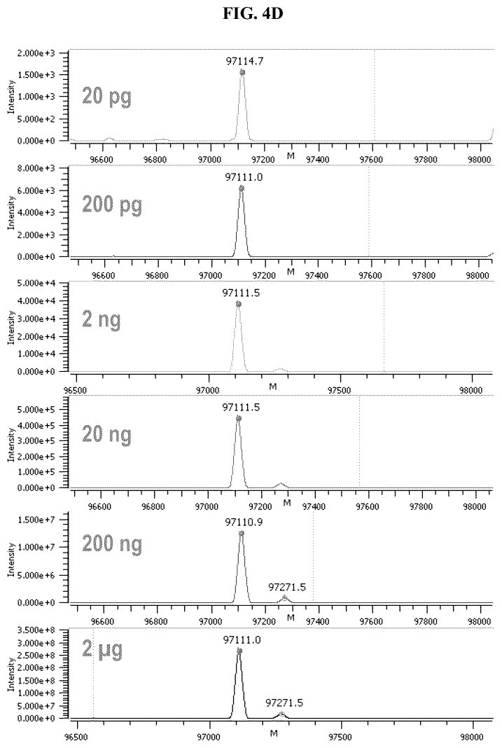

The LOD and limit of quantitation (LOQ) in neat solution were further assessed as shown in FIG. 4. The Fab₂ fragment of mAb1 was analyzed using native SCX-MS, with the Fab₂ fragment of mAb2 used as an internal standard, as shown in a TIC in FIG. 4A. FIG. 4B shows a comparison of the actual concentration of mAb1 compared to the intensity normalized to the internal standard as measured by the method of the present invention, at a range of concentrations between 20 pg and 20 ng. The actual versus measured concentrations show a linear relationship with a weighted R² of 0.9954, demonstrating the ability of the method of the present invention to accurately and sensitively quantitate an analyte at low concentrations. FIG. 4C shows the same comparison made with a range of concentrations between 20 ng and 2 μg, with a strong linear relationship demonstrated again at this higher concentration range. Exemplary mass spectra between 20 pg and 2 μg are shown in FIG. 4D, further illustrating the sensitivity and specificity of the method of the present invention.

### Example 4. Establishing Limit of Detection and Limit of Quantitation in Serum

The robustness of the method of the present invention was further demonstrated using analytes from a mouse serum sample. Analysis of a protein of interest in serum presents numerous additional challenges, including heterogeneity of the protein of interest due to biotransformation, and interference due to a complex matrix, such as high concentration serum proteins.

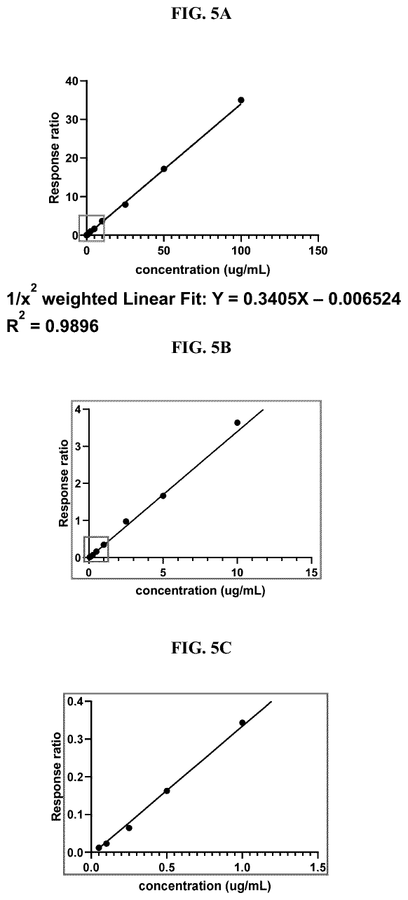

Fab₂ fragments of mAb1 were prepared as previously described, and subjected to native SCX-MS analysis. The linearity of the response ratio (the measured analyte intensity normalized to an internal standard) to actual concentration of the antibody is shown in FIG. 5A. FIG. 5B and FIG. 5C show further insets, demonstrating the linearity of the response even at low concentrations. These results demonstrate the sensitivity and effectiveness of the method of the present invention in quantifying antibodies even at low concentrations in serum.

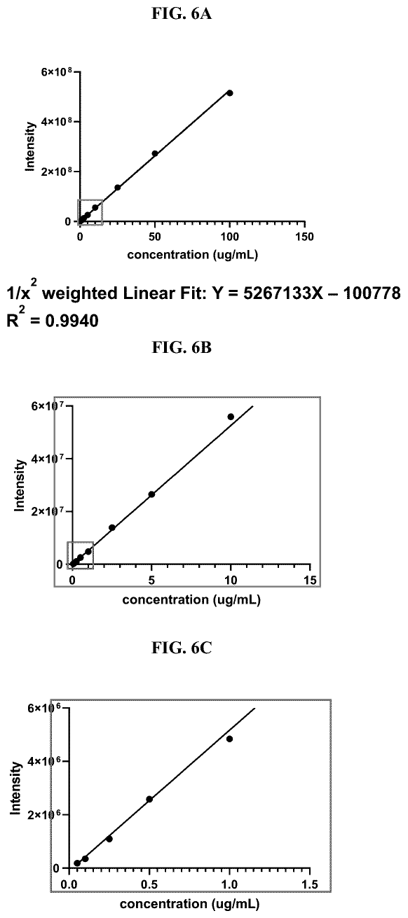

The stability of the method of the present invention was further demonstrated by plotting the linearity of the measured intensity, without normalization to an internal standard, compared to antibody concentration, as shown in FIG. 6A. FIGS. 6B and 6C show further insets demonstrating the linearity of measured intensity at low concentrations in serum, even without normalization to an internal standard.

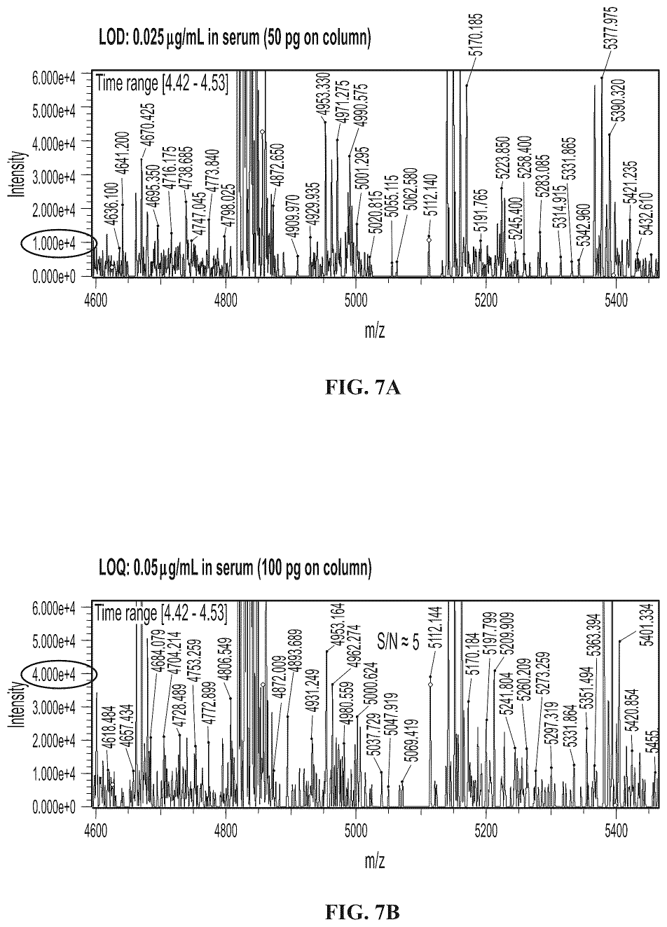

Mass spectra illustrating the LOD and LOQ of mAb1 Fab₂ in serum and in neat solution are shown in FIG. 7. The LOD was determined to be as low as 0.025 μg/mL in serum, which is equivalent to 50 pg on the SCX column, as shown in FIG. 7A. The LOQ was determined to be as low as 0.05 μg/mL in serum, which is equivalent to 100 pg on the SCX column, as shown in FIG. 7B. A signal-to-noise (S/N) ratio of 5 is indicated as a reasonable standard for establishing the LOQ. The absolute intensities of mAb1 Fab₂ detected from serum samples were higher than those detected in neat solution, suggesting that the limit of sensitivity of serum samples is due to noise from co-Wed serum protein.

In addition to the examples disclosed herein, even lower LOD and LOQ are possible using the method of the present invention in more favorable conditions that would be known to a person of skill in the art, for example using an antibody with a later elution time, or using greater washing volume during IP.

### Example 5. Materials and Methods for Native SEC-PCD-MS Analysis

Deionized water was provided by a Milli-Q integral water purification system installed with a MilliPak Express 20 filter (Millipore Sigma, Burlington, MA, Cat. NO. MPGP02001). Ammonium acetate (LC/MS grade) was purchased from Sigma-Aldrich (St. Louis, MO, Prod. No. 73594). Peptide N-glycosidase F(PNGase F) was purchased from New England Biolabs Inc (Ipswich, MA, Prod. No. P0704L). FabRICATOR® (IdeS) was purchased from Genovis (Cambridge, MA, Prod. No. AO-FR1-250). Invitrogen UltraPure 1 M Tris-HCl buffer, pH 7.5 (Ref. No. 15567-027), Pierce™ DTT (Dithiothreitol, No-Weigh™ Format, Ref. No. A39255), and Acetonitrile (ACN; Optima LC/MS grade, Prod. No. A955-4) were purchased from Thermo Fisher Scientific (Waltham, MA). Formic acid (FA, 98-100%, Suprapur for trace metal analysis) was purchased from Millipore Sigma (Burlington, MA, Prod. No. 1.11670.0250). 2-propanol (IPA; HPLC grade) was purchased from Sigma Aldrich (St. Louis, MO, Prod. No. 65-0447-4L).

Native SEC chromatography was performed on an UltiMate 3000 UHPLC System (Thermo Fisher Scientific, Bremen, Germany) equipped with an Acquity BEH200 SEC column (4.6×300 mm, 1.7 μm, 200 Å; Waters, Milford, MA) with the column compartment set to 30° C. An isocratic flow of 150 mM ammonium acetate at 0.2 mL/min was applied. To enable post-column denaturation, a denaturing solution consisting of 60% ACN, 36% water, and 4% FA was delivered by a secondary pump at a flow rate of 0.2 mL/min and then mixed with the SEC eluate (1:1 mixing) using a T-mixer before being subjected to MS detection. To enable online native MS analysis, the combined analytical flow (0.4 mL/min) was split into a microflow (<10 μL/min) for nano-electrospray ionization (NSI/nESI)-MS detection and a remaining high flow for UV detection. A Thermo Q Exactive UHMR (Thermo Fisher Scientific, Bremen, Germany) equipped with a Microflow-Nanospray Electrospray Ionization (MnESI) Source and a Microfabricated Monolithic Multi-nozzle (M3) emitter (Newomics, Berkley, CA) was used for native MS analysis. To disable PCD, the flow of the denaturing solution was set to zero.

Intact mass spectra from nSEC-MS analysis under native or PCD conditions were deconvoluted using Intact Mass™ software from Protein Metrics.

### Example 5. IP-nSEC-PCD-MS Method for Characterizing Biotransformation Products of Multispecific mAbs

Multispecific mAbs need to be assessed for in vivo behaviors during developability assessment. For example, the structural integrity at the G45 linker regions in an in vivo environment must be evaluated. Additionally, interactions with other serum proteins in the crowded serum environment should be characterized. In order to sensitively and efficiently characterize multispecific mAbs in pharmacokinetic samples, an additional method was developed including immunoprecipitation, size exclusion chromatography, post-column denaturation, and mass spectrometry, operated under near-native conditions.

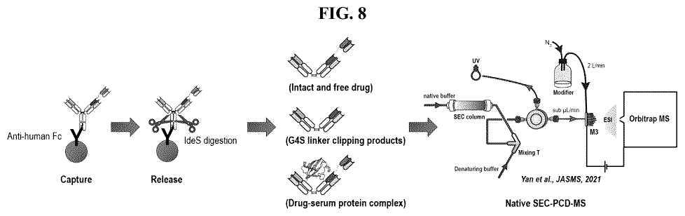

A workflow of the IP-nSEC-PCD-MS method of the present invention is illustrated in FIG. 8. Immunoprecipitation and digestion of an antibody, for example a multispecific antibody, from a biological sample, for example a mouse PK sample, may be performed as described in Example 1. Antibody fragments and complexes derived following IdeS digestion may include, for example, intact and free drug; G4S linker clipping products; and drug-serum protein complex. The varying protein species and complexes may then be subjected to SEC under native conditions, subjected to post-column denaturation to separate non-covalently bound species, and then subjected to MS analysis to identify and/or quantify protein species and complexes. Taking advantage of a previously described nLC-MS platform that can accommodate a high flow rate (for example, up to 0.8 mL/minute), integration of PCD with nSEC-MS can be readily achieved by introducing a post-column denaturant flow (for example, about 0.2 mL/minute) to the nSEC flow (for example, about 0.2 mL/minute) using a T-mixer.

The denaturing solvent was carefully selected based on two primary considerations. First, the final flow after post-column mixing should still be highly compatible with direct MS detection. Second, because of the short denaturation time (e.g., less than 1 second from the T-mixer to MS), the desired denaturing solvent should be capable of disrupting the majority of the non-covalent interactions instantaneously after post-column mixing. After evaluating a series of denaturing solvent systems containing varying levels of acetonitrile (ACN) and formic acid (FA), an optimized formula comprised of 60% ACN, 4% FA, and 36% water was selected for PCD application.

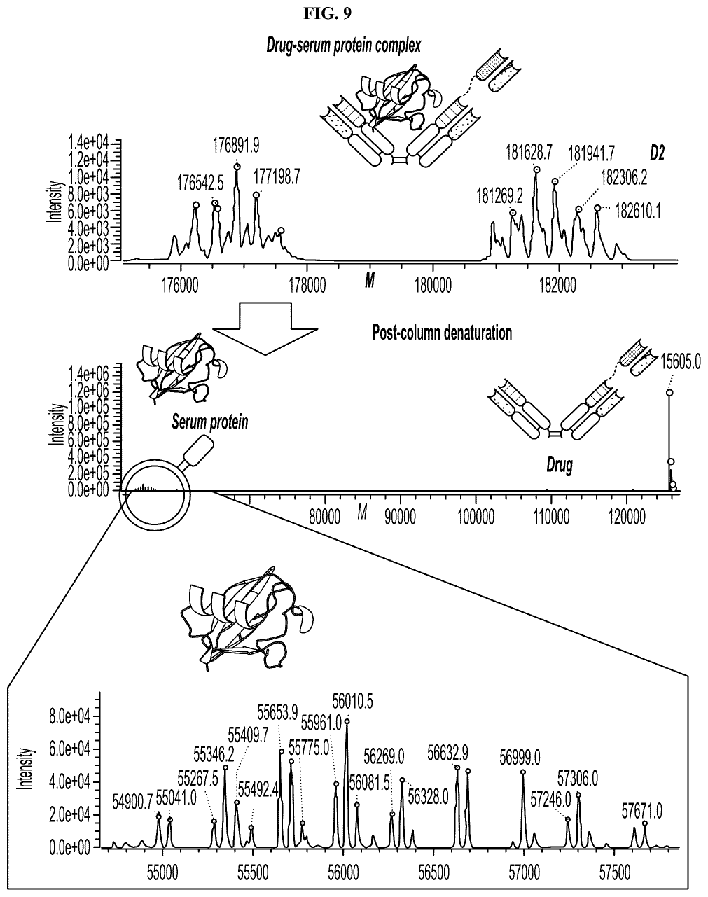

nSEC separation allows for the separation of unbound drug and unbound serum protein from bound drug and serum protein, while subsequent post-column denaturation allows for the separation of the non-covalently bound drug-serum protein complex and sensitive detection of each component, as shown in FIG. 9. Protein species analyzed using the described method all exhibit “native-like” mass spectra under the selected PCD conditions, which is a highly desirable feature because it reduces the spectral overlapping from multiple species that are simultaneously dissociated from the same complexes and detected in the same MS scan. Compared to typical ESI-MS spectra under denaturing conditions, “native-like” spectra exhibit much fewer charge states and greater spatial resolution, making them easier to interpret and process, for example for generating extracted ion chromatograms.

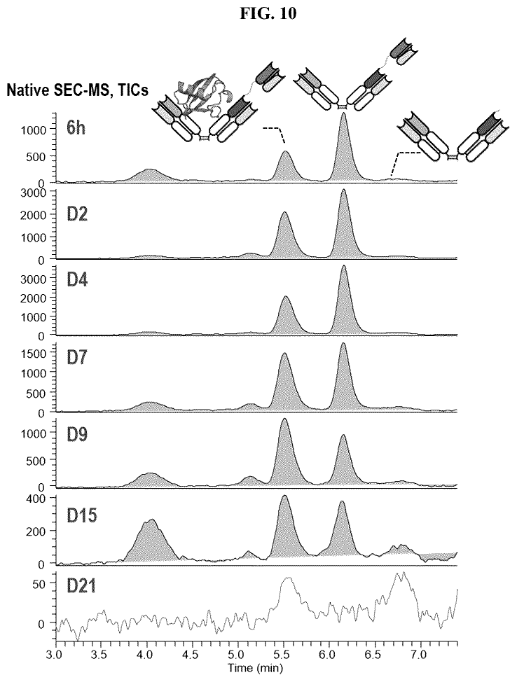

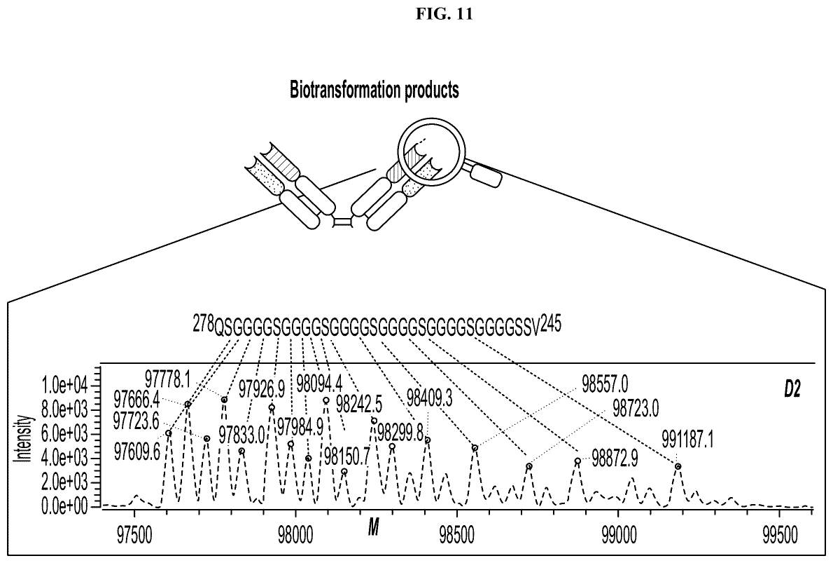

Using the IP-nSEC-PCD-MS method of the present invention, biotransformation of an antibody, for example a therapeutic trispecific antibody, can be characterized over time after administration to a subject. FIG. 10 shows a change in the relative abundance of antibody species as a function of time after administration to a subject, as measured by total ion chromatograms (TICs) from mouse PK samples. Over time, the relative abundances of drug-serum protein complex and G4s linker clipping products increase compared to intact and free drug. The specific identity and sequence of biotransformation products can be sensitively identified, as shown in FIG. 11.

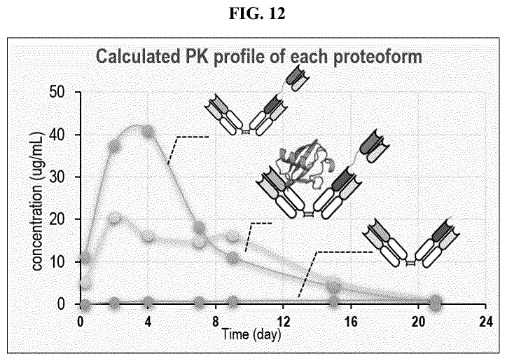

Using the IP-nSEC-PCD-MS method of the present invention, the pharmacokinetic profile of each proteoform of the protein of interest can be calculated and compared, as shown in FIG. 12. Operated under near native conditions, this approach not only exhibits high-resolution separation of biotransformation products, but also preserves drug-serum protein interactions, allowing for detailed characterization of in vivo processes after administration of a therapeutic protein to a subject.

What is claimed is:

1. A method for identifying, quantifying, and/or characterizing a protein of interest, comprising:
(a) contacting a sample including a protein of interest to a solid surface to form an immobilized protein of interest;
(b) eluting said immobilized protein of interest or a fragment thereof to form an enriched protein of interest or fragment thereof;
(c) subjecting said enriched protein of interest or fragment thereof to size exclusion chromatography under native conditions to form a size exclusion chromatography eluate;
(d) contacting said size exclusion chromatography eluate to a denaturing solution to form a denatured eluate; and
(e) subjecting said denatured eluate to mass spectrometry analysis to identify, quantify, and/or characterize said protein of interest.

2. The method of claim 1, wherein said sample is selected from a group consisting of cell culture fluid, harvested cell culture fluid, drug substance, drug product, a tissue sample, blood, serum, saliva, or urine.

3. The method of claim 2, wherein said sample is human serum or mouse serum.

4. The method of claim 1, wherein said protein of interest is selected from a group consisting of a recombinant protein, a therapeutic protein, an antibody, a bi specific antibody, a trispecific antibody, a multispecific antibody, an antibody fragment, a fusion protein, a trap protein, a single-chain variable fragment, and combinations thereof.

5. The method of claim 1, wherein said protein of interest is a therapeutic antibody.

6. The method of claim 1, wherein said solid surface is selected from a group consisting of a microplate, resin, agarose beads, or magnetic beads.

7. The method of claim 1, wherein said solid surface is adhered to an antibody that can specifically bind to said protein of interest.

8. The method of claim 7, wherein said adhering is mediated by biotin and avidin or streptavidin.

9. The method of claim 7, wherein said antibody is an anti-Fc antibody.

10. The method of claim 1, further comprising subjecting said immobilized protein of interest to at least one washing step to remove non-specifically bound components.

11. The method of claim 1, further comprising contacting said immobilized protein of interest to at least one digestive enzyme.

12. The method of claim 11, wherein said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease, LysC endoproteinase, endoproteinase AspN, endoproteinase GluC, outer membrane protein T, immunoglobulin-degrading enzyme of Streptococcus pyogenes (IdeS), thermolysin, papain, pronase, V8 protease, active fragments thereof, homologs thereof, variants thereof, and combinations thereof.

13. The method of claim 12, wherein said at least one digestive enzyme comprises IdeS or a variant thereof.

14. The method of claim 1, wherein eluting said immobilized protein of interest or fragment thereof comprises subjecting said solid surface to centrifugation.

15. The method of claim 1, wherein said fragment is selected from the group consisting of a Fab fragment, a Fab′ fragment, a Fab₂ fragment, a F(ab′)2 fragment, a Fv fragment, a Fd′ fragment, and a Fd fragment.

16. The method of claim 1, wherein said size exclusion chromatography system is coupled to said mass spectrometer.

17. The method of claim 1, wherein a mobile phase for said size exclusion chromatography comprises ammonium acetate, ammonium bicarbonate, ammonium formate, or a combination thereof.

18. The method of claim 1, wherein a mobile phase for said size exclusion chromatography comprises from 100 mM to 200 mM ammonium acetate, or about 150 mM ammonium acetate.

19. The method of claim 1, wherein said denaturing solution comprises acetonitrile, formic acid, or a combination thereof.

20. The method of claim 1, wherein said denaturing solution comprises from 40% to 80% acetonitrile.

21. The method of claim 1, wherein said denaturing solution comprises from 1% to 10% formic acid.

22. The method of claim 1, wherein said denaturing solution comprises about 60% acetonitrile and about 4% formic acid.

23. The method of claim 1, wherein said denaturing solution is contacted to said size exclusion chromatography eluate using a T-mixer.

24. The method of claim 1, wherein a flow from said size exclusion chromatography system is from 0.1 mL/minute to 0.5 mL/minute or about 0.2 mL/minute.

25. The method of claim 1, wherein a flow of said denaturing solution is from 0.1 mL/minute to 0.5 mL/minute or about 0.2 mL/minute.

26. The method of claim 1, wherein said mass spectrometer is an electrospray ionization mass spectrometer, nano-electrospray ionization mass spectrometer, or a triple quadrupole mass spectrometer.

27. The method of claim 1, wherein a flow splitter is used to couple said size exclusion chromatography system with said mass spectrometer and a detector.

28. The method of claim 27, wherein said detector is an ultraviolet detector or a fluorescence detector.

29. The method of claim 27, wherein a flow from said size exclusion chromatography system is split into a low flow to said mass spectrometer and a high flow to said detector.

30. The method of claim 29, wherein said low flow is less than 20 μL/minute or less than 10 μL/minute.

31. A method for identifying, quantifying, and/or characterizing at least one interacting protein in a sample that interacts with a protein of interest, comprising:
(a) contacting a sample including at least one complex of at least one interacting protein and a protein of interest to a solid surface to form an immobilized complex;
(b) eluting said immobilized complex to form an enriched complex;
(c) subjecting said enriched complex to size exclusion chromatography under native conditions to form a size exclusion chromatography eluate;
(d) contacting said size exclusion chromatography eluate to a denaturing solution to form a denatured eluate; and
(e) subjecting said denatured eluate to mass spectrometry analysis to identify, quantify, and/or characterize said at least one interacting protein.

32. The method of claim 31, wherein said at least one interacting protein comprises a serum protein.

33. A method for identifying, quantifying, and/or characterizing at least one biotransformation product of a protein of interest, comprising:
(a) contacting a sample including at least one biotransformation product of a protein of interest to a solid surface to form an immobilized biotransformation product;
(b) eluting said immobilized biotransformation product or a fragment thereof to form an enriched biotransformation product or fragment thereof;
(c) subjecting said enriched biotransformation product or fragment thereof to size exclusion chromatography under native conditions to form a size exclusion chromatography eluate;
(d) contacting said size exclusion chromatography eluate to a denaturing solution to form a denatured eluate; and
(e) subjecting said denatured eluate to mass spectrometry analysis to identify, quantify, and/or characterize said at least one biotransformation product.

34. The method of claim 33, wherein said at least one biotransformation product comprises a post-translational modification, truncation, aggregate, fragment, degradation product, or combination thereof.

35. The method of claim 33, wherein said protein of interest comprises a linker and said at least one biotransformation product comprises a clipped form of said linker.

36. A method for producing a pharmacokinetic profile of a protein of interest, comprising:
(a) quantifying a concentration of said protein of interest at a first time point after administration of said protein of interest to a subject by:
(i) obtaining a sample from said subject including said protein of interest at a first time point after administration of said protein of interest to said subject; and
(ii) quantifying said protein of interest according to the method of claim 1; and
(b) repeating step (a) for at least one additional time point to produce a pharmacokinetic profile of said protein of interest.

37. The method of claim 36, wherein said pharmacokinetic profile includes at least one biotransformation product of said protein of interest.

38. The method of claim 36, wherein said pharmacokinetic profile includes at least one interacting protein that interacts with said protein of interest.
